(19) United States
(12) Patent Application Publication
(10) Pub. No.: US 2007/0015708 A1
(43) Pub. Date: Jan. 18, 2007

(54) Methods and compositions for inhibiting tumor growth and angiogenesis

(76) Inventor: Erkki Ruoslahti, Rancho Santa Fe, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/521,715

(22) Filed: Sep. 15, 2006

Related U.S. Application Data
(62) Division of application No. 10/431,642, filed on May 5, 2003.
Application No. 10/431,642 is a continuation-in-part of application No. 10/005,171, filed on Dec. 3, 2001, now abandoned.
(60) Provisional application No. 60/331,357, filed on Dec. 4, 2000.

(51) Int. Cl. A61K 38/54

(52) U.S. Cl. 514/12

(57) ABSTRACT

The invention provides compositions comprising angiogenesis inhibitors and RGD-containing plasma adhesion proteins in a pharmaceutical carrier. This invention also provides methods of inhibiting angiogenesis, tumor growth and metastasis by administering angiogenesis inhibitors in combination with RGD-containing plasma adhesion proteins in a pharmaceutical carrier.

### CROSS REFERENCE TO RELATED APPLICATIONS

This application is a divisional of U.S. application Ser. No. 10/431,642, filed May 5, 2003, which is a continuation-in-part of U.S. application Ser. No. 10/005,171, filed Dec. 3, 2001, which claims the benefit under 35 U.S.C. § 119(e) to U.S. Provisional Application No. 60/331,357, filed Dec. 4, 2000, which was converted from U.S. Ser. No. 09/729,657, all of which are hereby incorporated reference.

### GOVERNMENTAL INTEREST

This invention was made with government support under grant number CA74238 and the Cancer Center Support Grant CA30199 awarded by the National Cancer Institute and grant DAMD17-00-1-0556 awarded by the Department of Defense. The United States Government may have certain rights in this invention.

This work was also supported by grants DAMD17-00-1-0556 from the Department of Defense, and CA88420 and Cancer Center Support Grant CA30199 from the National Cancer Institute.

### SEQUENCE LISTING

The present application is being filed along with duplicate copies of a CD-ROM marked “Copy 1” and “Copy 2” containing a Sequence Listing in electronic format. The duplicate copies of the CD-ROM each contain a file entitled BURNHAM.8CP1DV1.TXT created on Sep. 15, 2006 and is 17,715 bytes in size. The information on these duplicate CD-ROMs is incorporated herein by reference in its entirety.

### BACKGROUND OF THE INVENTION

1. Field of the Invention

This invention relates generally to the field of cancer biology and, more specifically to compositions and methods for inhibiting angiogenesis, tumor growth, and metastasis.

2. Description of the Related Art

This year about 556,500 Americans are expected to die of cancer, an average of more than 1,500 people per day. Cancer is the second leading cause of death in the United States, where one out of every four deaths is due to cancer. Since 1990, approximately 13 million new cases have been diagnosed and nearly five million lives have been lost to cancer. In 2003, an estimated 1,334,100 new cancer cases will be diagnosed. While progress in preventing and treating cancer has been made, including particular success against Hodgkin's lymphoma and certain other forms, many types of cancer remain substantially impervious to prevailing treatment protocols.

One of the hallmarks of cancer, as well as that of over seventy other diseases, including diabetic blindness, age-related macular degeneration, rheumatoid arthritis and psoriasis, is the body's loss of control over angiogenesis. Angiogenesis-dependent diseases result when new blood vessels either grow excessively or insufficiently. Excessive angiogenesis occurs when diseased cells produce and release abnormal amounts of angiogenic growth factors, overwhelming the effects of natural angiogenesis inhibitors. The resulting new blood vessels feed diseased tissues, which in turn destroy normal tissues.

Upon their release, angiogenic growth factors diffuse into nearby tissues and bind to specific receptors located on the endothelial cells of nearby preexisting blood vessels. Once growth factors bind to their receptors, the endothelial cells become activated and send signals from the cell surface to the nucleus. As a result, the endothelial cell's machinery begins to produce new molecules including enzymes that create tiny holes in the basement membrane that surrounds existing blood vessels. As the endothelial cells begin to proliferate, they migrate out through the enzyme-created holes of the existing blood vessel towards the diseased tissue; in the case of cancer, the endothelial cells migrate towards the tumor. Specialized molecules called adhesion molecules or integrins provide anchors that allow the new blood vessel to sprout forward. Additional enzymes, among them matrix metalloproteinases (MMPs), are produced to dissolve the tissue in front of the growing blood vessel tip to allow for its continued tissue invasion. As the vessel extends, the tissue is remolded around the vessel and endothelial cells roll up to form a new blood vessel. Subsequently, individual blood vessels connect to form blood vessel loops that can circulate blood. Finally, the newly formed blood vessels are stabilized by specialized muscle cells (smooth muscle cells, pericytes) that provide structural support and blood flow through the neovascularized tissue begins.

Significantly, angiogenesis is one of the critical events required for cancer metastasis. Metastasis, the ability of cancer cells to penetrate into lymphatic and blood vessels, circulate through the bloodstream, and invade and grow in normal tissues elsewhere makes cancer a life-threatening disease. Tumor angiogenesis is the proliferation of a network of blood vessels that penetrates into cancerous growths, supplying nutrients and oxygen and removing waste products.

A growing class of anti-angiogenic substances is derived from extracellular matrix and blood proteins by proteolysis or other modifications. These substances include fragments from thrombospondin (Good et al., Proc. Natl. Acad. Sci. USA 87:6624-6628 (1990)), plasminogen (angiostatin; O'Reilly et al., Cell 79:315-328 (1994)), collagen type XVIII (endostatin; O'Reilly et al., Cell 88:277-285 (1997)), collagen type XVIII (endostatin; O'Reilly et al., supra (1997)), collagen type IV (tumstatin; Maeshima et al., Science 295:140-143 (2002)), a modified form of aniithrombin III (O'Reilly et al., Science 285:1926-1928, (1999)), and the fibronectin fragment anastellin (Pasqualini et al., Nature Med. 2:1197-1203 (1996); Yi and Ruoslahti, Proc. Natl. Acad. Sci USA 98:620-624 (2001)). These substances also include synthetic β-sheet compound, anginex (Mayo et al, Angiogenesis 4:45-51 (2001)), and the matricellular protein, SPARC (Chlenski et al., Cancer Res. 62:7357-7363 (2002)). The molecular mechanisms whereby these substances exert their anti-angiogenic activities are unknown.

Various anti-angiogenic proteins share certain binding activities. Anastellin binds to and polymerizes fibronectin and fibrinogen (Morla and Ruoslahti, J. Cell Biol. 118:421-429, (1992); Morla et al., Nature 367:193-196 (1994)). The anti-angiogenic form of antithrombin III (henceforth referred to as antithrombin) is similar to the modified antithrombin that binds vitronectin (Ill and Ruoslahti, supra (1985); deBoer et al., J. Biol. Chem. 267:2264-2268 (1992)). Fibronectin and vitronectin (Tomasini and Mosher, Prog Hemost Thromb 10:269-305 (1991)) contain the RGD cell attachment sequence recognized by many of the integrin family cell adhesion receptors (Ruoslahti, Ann. Rev. Cell Dev. Biol. 12:697-715 (1996); RGD is an abbreviation of the amino acid sequence arginine-glycine-aspartate). The RGD sequence is also present in several other extra-cellular matrix and blood proteins, such as various collagens, thrombospondin fibrinogen and laminin. Anastellin and antithrombin are not the only angiogenesis inhibitors to interact with adhesion proteins: angiostatin, and its parent protein plasminogen, bind vitronectin (Kost et al., Eur. J. Biochem. 236:682-688 (1996)), endostatin binds fibulins and nidogen-2 (Miosge et al., FASEB J. 13:1743-1750 (1999)). In addition, each of these anti-angiogenic proteins bind to heparin and heparan sulfate. These shared binding activities suggest a common mechanism of action.

Anti-angiogenic therapies, aimed at destroying newly formed blood vessels and halting new blood vessel growth, are needed to treat cancer as well as other conditions characterized by excessive angiogenesis. In the case of cancer, there exists a particular need to supplement existing methods of treating cancer with anti-angiogenic therapies aimed at halting angiogenesis, tumor growth and metastasis.

Some cancer patients who have received chemotherapy have low fibronectin levels (Choate and Mosher, Cancer 51:1142-1147 (1983)). Because the anti-angiogenic activity of anastellin and endostatin require the presence of plasma fibronectin, these angiogenesis inhibitors may not be effective in patients who have received chemotherapy and as a result have low fibronectin levels. Such individuals might be excluded from receiving endostatin or anastellin, or the anti-angiogenic protein might be given together with fibronectin. Similarly, when antithrombin treatment is contemplated this substance might be given together with vitronectin.

The present invention satisfies the need to supplement existing methods of treating cancer with anti-angiogenic therapies aimed at halting angiogenesis, tumor growth and metastasis, and provides related advantages as well.

### SUMMARY OF THE INVENTION

The invention described herein relates to angiogenesis inhibitors in conjunction with plasma adhesion proteins.

Accordingly, one embodiment of the invention relates to a substantially pure composition comprising an angiogenesis inhibitor and an RGD-containing plasma adhesion protein in a pharmaceutically acceptable carrier. The angiogenesis inhibitor can comprise anastellin. Further, the RGD-containing plasma adhesion protein can comprise fibronectin. Alternatively, the angiogenesis inhibitor can comprise antithrombin. In addition, the RGD-containing plasma adhesion protein can comprise vitronectin. In another embodiment, the angiogenesis inhibitor can comprise endostatin. The RGD-containing plasma adhesion protein can comprise fibronectin. In still another embodiment, the angiogenesis inhibitor can comprise anginex. Further, the RGD-containing plasma adhesion protein can comprise fibronectin.

Another embodiment of the invention provides a method of inhibiting angiogenesis in a patient, comprising providing a patient in need of angiogenesis-inhibiting treatment; and administering to said patient an angiogenesis inhibitor and an RGD-containing plasma adhesion protein in a pharmaceutically acceptable carrier. The angiogenesis inhibitor can comprise anastellin and the RGD-containing plasma adhesion protein can comprise fibronectin. In another embodiment, the angiogenesis inhibitor can comprise antithrombin and the RGD-containing plasma adhesion protein can comprise vitronectin. In yet another embodiment, the angiogenesis inhibitor can comprise endostatin and the RGD-containing plasma adhesion protein can comprise fibronectin. In still another embodiment, the angiogenesis inhibitor can comprise anginex and the RGD-containing plasma adhesion protein can comprise fibronectin.

Still another embodiment of the invention provides a method of inhibiting angiogenesis in a patient; comprising providing a patient in need of angiogenesis-inhibiting treatment; determining the level of plasma adhesion protein in said patient, and administering to said patient an angiogenesis inhibitor that is activated by said plasma adhesion protein. The angiogenesis inhibitor can comprise anastellin and the plasma adhesion protein can comprise fibronectin. In another embodiment, the angiogenesis inhibitor can comprise antithrombin and the plasma adhesion protein can comprise vitronectin. In still another embodiment, the angiogenesis inhibitor can comprise endostatin and the plasma adhesion protein can comprise fibronectin. In yet another embodiment, the angiogenesis inhibitor can comprise anginex and the plasma adhesion protein can comprise fibronectin.

Yet another embodiment of the invention provides a method of treating cancer in a patient, comprising providing a patient in need of treatment of a tumor; and administering to said patient an angiogenesis inhibitor and an RGD-containing plasma adhesion protein in a pharmaceutically acceptable carrier. The angiogenesis inhibitor can comprise anastellin and the RGD-containing plasma adhesion protein can comprise fibronectin. In another embodiment, the angiogenesis inhibitor can comprise antithrombin and the RGD-containing plasma adhesion protein can comprise vitronectin. In still another embodiment, the angiogenesis inhibitor can comprise endostatin and the RGD-containing plasma adhesion protein can comprise fibronectin. Alternatively, the angiogenesis inhibitor can comprise anginex and the RGD-containing plasma adhesion protein can comprise fibronectin.

### BRIEF DESCRIPTION OF THE DRAWINGS

FIG. 1 is a bar chart that shows the effect of systemic treatment with anastellin (SEQ ID NO: 1) on the growth of blood vessels in human tumors xenografted into mice.

FIG. 2 is a bar chart that shows that anastellin lacks anti-angiogenic activity in plasma fibronectin-deficient mice but is active in vitronectin null mice. Shown are the number of blood vessels (FIGS. 2A and C) and hemoglobin content (FIGS. 2B and D) in matrigel plugs removed from fibronectin-deficient mice (pFN−) and their normal littermates (pFN+) (FIGS. 2A and B), or vitronectin null (VN null) mice and their wild-type (wt) controls (FIGS. 2C and D). The mice were treated daily with intraperitoneal injections of anastellin or PBS. The brackets and the P values show the significance level of the differences observed between the indicated test groups. NS=not significant.

FIG. 3 is a bar chart that shows that antithrombin is active in plasma fibronectin-deficient mice but is inactive in vitronectin null mice. Mice with matrigel plugs were treated with antithrombin or PBS as in FIG. 2. The number of blood vessels (FIG. 3A, C) and hemoglobin content (FIGS. 3B and D) in the plugs removed from fibronectin-deficient mice (pFN−) and their normal littermates (pFN+) (FIGS. 3A and B); or vitronectin null (VN null) mice and their wild-type (wt) controls (FIGS. 3C and D) are shown. The results from 48 mice were pooled. The brackets and the P values show the significance level of the differences observed between the indicated test groups. NS=not significant.

FIG. 4 is a bar chart that shows that endostatin lacks anti-angiogenic activity in plasma fibronectin-deficient mice. Fibronectin-deficient mice (pFN−) and their wild type littermates (pFN+) were implanted with matrigel plugs and systemically treated with endostatin or PBS as in FIG. 2. The number of blood vessels (FIG. 4A) and hemoglobin content (FIG. 4B) in the plugs are shown. The results from 24 mice were pooled. The brackets and the P values show the significance level of the differences observed between the indicated test groups. NS=not significant.

FIG. 5 is a line graph that shows that addition of anginex to a solution of fibronectin causes the formation of insoluble protein complexes.

### DETAILED DESCRIPTION OF THE PREFERRED EMBODIMENT

Embodiments of the invention relate to the discovery that angiogenesis inhibitors function as anti-tumor agents in conjunction with plasma adhesion proteins. Accordingly, embodiments of the invention include substantially pure compositions comprising protein inhibitors of angiogenesis and plasma adhesion proteins in a pharmaceutically acceptable carrier. Angiogenesis inhibitors contemplated for use in this invention include, but are not limited to, anastellin (SEQ ID NO: 1), antithrombin (SEQ ID NO: 2), endostatin (SEQ ID NO: 3), and anginex (SEQ ID NO: 4).

In one embodiment, the invention provides a composition of anastellin (SEQ ID NO: 1) and fibrinogen or fibronectin. In another embodiment, the invention provides a composition of antithrombin (SEQ ID NO 2; NCBI accession number P01008) and vitronectin. In still another embodiment, the invention provides a composition of endostatin (SEQ ID NO: 3; NCBI accession number P39060) and fibronectin. In yet another embodiment, the invention provides a composition of anginex, a synthetic anti-angiogenic peptide of amino acid sequence: ANIKLSVQMK LFKRHIKWKI IVKLNDGREL SLD (SEQ ID NO: 4), and fibronectin.

Embodiments of the invention include methods of inhibiting angiogenesis, tumor growth, and metastasis through administration of substantially pure compositions comprising protein inhibitors of angiogenesis and plasma adhesion proteins. In one embodiment, the plasma adhesion proteins are RGD-containing plasma adhesion proteins.

In another embodiment, methods of determining a treatment for a tumor is provided by measuring the level of plasma adhesion proteins in a patient. With this data, a physician can more easily determine which type of angiogenesis inhibitor would be more effective to treat a tumor. For example, the determination that a patient has low levels of the plasma adhesion protein fibronectin would lead a physician to choose antithrombin as an angiogenesis inhibitor.

In one embodiment the invention provides a method of inhibiting angiogenesis, tumor growth, and metastasis through the administration of anastellin and fibrinogen. In another embodiment the invention provides a method of inhibiting tumor growth, angiogenesis, and metastasis through the administration of antithrombin and vitronectin. In still another embodiment the invention provides a method of inhibiting tumor growth, angiogenesis, and metastasis through the administration of endostatin and fibronectin. In yet another embodiment the invention provides a method of inhibiting tumor growth, angiogenesis, and metastasis through the administration of anginex and fibronectin. Such administration can be in vivo, ex vivo, or in vitro.

As used herein, the term “anastellin” refers to an amino acid fragment of the first type III fibronectin repeat that is about 76 amino acids in length and designated herein as SEQ ID NO: 1. The anastellin peptide spans residues 600 to 674 of fibronectin according to the numbering of Komblihtt et al., EMBO J. 4(7):1755-9 (1985), which is incorporated herein by reference, and has the following sequence: NAPQPSHISK YILRWRPKNS VGRWKEATIP GHLNSYTIKG LKPGVVYEGQ LISIQQYGHQ EVTRFDFTTT STSTP (SEQ ID NO: 1). Functionally, anastellin is an inhibitor of tumor growth, tumor angiogenesis and metastasis. Anastellin also functions as a fibronectin polymerizing agent and a fibrinogen polymerizing agent.

As used herein, the term “antithrombin” refers to antithrombin III (SEQ ID NO: 2) treated to become anti-angiogenic in ways that include denaturation or proteolytic cleavage by thrombin. The mature human anti-thrombin III (NCBI accession number P01008) is a 431 amino acid residue protein. Functionally, antithrombin is a vitronectin-dependent inhibitor of angiogenesis as shown herein.

As used herein, the term “endostatin” refers to a 182-amino acid fragment spanning residues 1334-1516 of the collagen alpha 1(XVIII) chain (SEQ ID NO: 3, NCBI accession number P39060). Functionally, endostatin is an inhibitor of tumor growth, tumor angiogenesis and metastasis, and functions in conjunction with fibronectin as shown herein.

As used herein, the term “anginex” refers to a synthetic β-sheet compound (Mayo et al, Angiogenesis 4:45-51 (2001)) that is about 33 amino acids in length, designated herein as SEQ ID NO: 4. The anginex peptide has the following sequence: ANIKLSVQMK LFKRHIKWKI IVKLNDGREL SLD (SEQ ID NO: 4). Functionally, anginex is an inhibitor of angiogenesis and tumor growth. Anginex also functions as a fibronectin polymerizing agent.

Anastellin, antithrombin, and endostatin are representative of a growing class of anti-angiogenic substances that can be derived from extracellular matrix and blood proteins by proteolysis or bther modifications well-known in the art. Anti-angiogenic substances also include, for example, but are not limited to, plasminogen (angiostatin; O'Reilly et al., supra (1994)) heparin-binding fragments of fibronectin (Homandberg et al., Am. J. Path. 120:327-332 (1985); Homandberg et al., Biochim. Biophys. Acta 874:61-71 (1986)), fragments from thrombospondin (Good et al., supra (1990)), and collagen type IV (tumstatin; Maeshima et al., supra (2002)). These anti-angiogenic substances also include the synthetic β-sheet compound, anginex (Mayo et al, supra (2001)) and the matricellular protein, SPARC (Chlenski et al., supra (2002)).

While the mechanism of activity of anti-angiogenic substances is unknown, the teachings regarding anastellin, antithrombin, endostatin and anginex provided herein elucidate a possible general mechanism of action for anti-angiogenic substances. These anti-angiogenic substances bind to one or more adhesion proteins: anastellin binds to and polymerizes fibronectin (Morla et al., supra (1994); Pasqualini et al., supra (1996)), the anti-angiogenic form of antithrombin III is similar to the modified antithrombin III that binds to vitronectin (Ill and Ruoslahti, supra (1985); deBoer et al., supra (1992)), endostatin has been shown to bind to fibulins and nidogen-2 (Miosge et al., supra (1999)). Moreover, angiostatin and its parent protein plasminogen can bind vitronectin (Kost et al., supra (1996)); Mulligan-Kehoe et al., J. Biol. Chem. 2:1197-1203 (2001); Tarui et al., J. Biol. Chem. 276:39562-39568 (2001)), as does SPARC (Rosenblatt et al., Biochem. J. 324:311-319 (1997)). Finally, as shown herein, the anti-angiogenic peptide anginex polymerizes fibronectin in a manner similar to anastellin, suggesting that the anti-angiogenic activity of anginex is also adhesion protein-dependent. These results suggest a common mechanism of action for protein inhibitors of angiogenesis: they form protein complexes with RGD-containing plasma adhesion proteins such as fibronectin or vitronectin, and these complexes are necessary for the anti-angiogenic activity. The targets of the complexes may be the αvβ3, α5β1 and α5β1 integrins, which are selectively expressed in angiogenic vessels.

Fibronectin, fibrinogen and each of the other ligands for the various anti-angiogenic substances described above, are adhesion proteins containing the RGD cell adhesion sequence as described by Ruoslahti, supra (1996), which is incorporated herein by reference. Moreover, these anti-angiogenic substances bind to the α5β1 and αvβ3 integrins, which is expressed at high levels in angiogenic endothelial cells and plays an important role in angiogenesis as described by Brooks et al., Science 264:569-571 (1994); Kim et al., J. Biol. Chem. 275:33920-33928 (2000), which are incorporated herein by reference. Direct binding endostatin to αv5β1 (Rehn et al., Proc. Natl. Acad. Sci. USA 98:1024-1029 (2001)), and the lack of tumstatin activity on cells that lack αvβ3 (Maeshima et al., supra (2002)), also suggest integrin involvement in the activities of anti-angiogenic proteins. Gene knockout experiments show that α5β1 is necessary for vascular development (Yang et al., Development 119:1093-1105 (1993)), although the vasculature develops and angiogenesis takes place in mice lack αvβ3 or all αv integrins (Reynolds et al., Nature Med. 8:27-34 (2002); Hynes, Nature Med. 8:918-921 (2002)). In an adult animal, perturbing the function of either α5β1 or αvβ3 causes endothelial cell apoptosis and inhibits angiogenesis (Brooks et al., supra (1994); Kim et al., Am. J. Pathol. 156:1345-1362 (2000); Cheresh and Stupack, Nature Med. 8:193-1934 (2002)). Moreover, synthetic RGD peptide polymers that mimic polymeric adhesion proteins can be effective inhibitors of angiogenesis (Saiki et al., Japan. J. Cancer Res. 81:668-675 (1990)). Therefore, it is likely that anti-angiogenic substances polymerize RGD-containing proteins in vivo, followed by binding of the polymers to the α5β1 and αvβ3 integrins on angiogenic endothelial cells, which leads to inhibition of cell proliferation and causes apoptosis.

One possibility is that the multimeric RGD-containing complexes generated by anti-angiogenic proteins perturb endothelial cell adhesion or affect cell polarity by binding to integrins on the luminal surface, causing sufficient disturbance to induce apoptosis. Alternatively, the complexes may be internalized by the angiogenic endothelial cells and may initiate apoptosis by releasing RGD-containing peptides into the cytoplasm (Buckley et al., Nature 397:534-539, 1999; Adderley and Fitzgerald, J. Biol. Chem. 275:5760-5766 (2000)). Another possibility is that the complexes could bind to bone marrow-derived endothelial cell precursors recruited to the site of angiogenesis and opsonize them for removal by phagocytic cells. A cell-opsonizing activity has been described for fibronectin in Saba and Cho, J. Reticulo Endoth. Soc. 22:583-596 (1977).

Fibronectin exists in two main forms: as an insoluble glycoprotein dimer that serves as a linker in the ECM and as a soluble disulphide linked dimer found in the plasma (plasma FN). While the plasma form is synthesized by hepatocytes, the ECM form is made by various other types of cells. As used herein, the term “superfibronectin” or “sFN” refers to multimers of fibronectin of high relative molecular mass, polymeric fibrillar forms of fibronectin and high molecular weight aggregates of fibronectin as described in Morla et al., supra (1994), which is incorporated herein by reference. Superfibronectin can be generated in vitro by treating purified fibronectin or fragments of fibronectin in solution with a fibronectin polymerizing agent such as anastellin as described in Morla et al., supra (1994), and in U.S. Pat. No. 5,922,676, which is incorporated herein by reference. Superfibronectin and anastellin inhibit angiogenesis and suppress tumor growth (Pasqualini et al., supra (1996); Yi and Ruoslahti, supra (2001)).

Embodiments of the invention include substantially pure compositions of angiogenesis inhibitors and RGD-containing plasma adhesion proteins. Preferred embodiments include anastellin and fibronectin, antithrombin and vitronectin, endostatin and fibronectin, and anginex and fibronectin. The substantially pure compositions described herein are useful for inhibiting angiogenesis, tumor growth and metastasis.

As used herein, the term “substantially pure” when used in reference to a composition is intended to mean that the composition is relatively free from cellular components or other contaminants that are not the desired composition, or its constituent polypeptides.

In addition, physiological buffers useful for in vivo administration are well-known in the art and further described below. The preparation of superfibronectin is known and described in the art (Pasqualini et al., supra (1996)) as well as described in Example I.

Anastellin, antithrombin, endostatin, anginex, fibronectin, vitronectin, fibrinogen, and their complexes are collectively referred to herein as examples of the constituent polypeptides of the invention. The constituent polypeptides are intended to encompass variants having substantially the same amino acid sequence as the reference constituent polypeptide and exhibit at least one of the functional activities thereof. An anastellin polypeptide of the invention can have the same amino acid sequence set forth in SEQ ID NO: 1. Alternatively, an anastellin polypeptide of the invention can have one or more amino acid alterations compared to the amino acid sequence set forth in SEQ ID NO: 1 that do not significantly change its biological activity. Similarly, antithrombin endostatin, anginex, and the fibronectin, vitronectin, and fibrinogen components of fibronectin and vitronectin complexes, respectively, can have either the same amino acid sequences or can have one or more alterations compared to the amino acid sequences set forth herein that do not significantly change the functional activities of the complexes.

An anastellin polypeptide useful for the compositions and methods of the invention can have substantially the same sequence as SEQ ID NO: 1 and can further be a polypeptide, fragment or segment having an identical amino acid sequence as SEQ ID NO: 1, or a polypeptide, fragment or segment having a similar, non-identical sequence that is considered by those skilled in the art to be a functional equivalent of SEQ ID NO: 1. Similarly, antithrombin, endostatin, and anginex, can further be a polypeptide, fragment or segment having an identical amino acid sequence as SEQ ID NO: 2, SEQ ID NO: 3, and SEQ ID NO: 4, respectively, or a polypeptide, fragment or segment having a similar, non-identical sequence that is considered by those skilled in the art to be a functional equivalent of SEQ ID NO: 2, SEQ ID NO: 3, and SEQ ID NO: 4, respectively.

Furthermore, a fibronectin polypeptide useful in the compositions and methods described herein can have substantially the same sequence as those known in the art and described in, for example, Kornblihtt et al., supra (1985), incorporated herein by reference, and can further be a polypeptide, fragment or segment having an identical amino acid sequence to one known in the art, or a polypeptide, fragment or segment having a similar, non-identical sequence that is considered by those skilled in the art to be a functional equivalent of fibronectin.

Likewise, a vitronectin polypeptide useful in the compositions and methods described herein can have substantially the same sequence as those known in the art, and can further be a polypeptide, fragment or segment having an identical amino acid sequence to one known in the art, or a polypeptide, fragment or segment having a similar, non-identical sequence that is considered by those skilled in the art to be a functional equivalent of vitronectin.

A functional equivalent of a constituent polypeptide retains at least one of the functional activities of its reference peptide. Functional activities of anastellin, antithrombin, endostatin, and anginex, include inhibition of angiogenesis, tumor growth and metastasis as well as the ability to polymerize both fibronectin or vitronectin in vitro and dependence of the presence of these proteins in vivo.

A functional equivalent of a constituent polypeptide of the invention such as anastellin, antithrombin, endostatin, anginex, fibronectin, or vitronectin includes those amino acid sequences that are sufficient for retention of a particular functional activity associated with the reference polypeptide. A functional equivalent of a constituent polypeptide of the invention can include those amino acid sequences sufficient for inhibition of angiogenesis, tumor growth or metastasis.

A constituent polypeptide of the invention can have at least 70%, at least 80%, at least 81%, at least 83%, at least 85%, at least 90%, at least 95% or more identity to the respective sequences of anastellin, antithrombin, endostatin, and anginex, set forth as SEQ ID NOS: 1, 2, 3, and 4 respectively. The constituent polypeptides of the invention also encompass modified forms of naturally occurring amino acids such as D-stereoisomers, non-naturally occurring amino acids, amino acid analogues, and mimetics so long as such polypeptides retain a functional activity of the reference polypeptide.

The constituent polypeptides include those polypeptides, fragments or segments having an amino acid sequence identical to that of the constituent polypeptide of the invention, or a polypeptide, fragment or segment having a similar, non-identical sequence that is considered by those skilled in the art to be a functional equivalent of the reference constituent polypeptide of the invention. Such a functional equivalent or functional fragment of a constituent polypeptide of the invention exhibits at least one functional activity of the reference polypeptide and can have, for example, at least 6 contiguous amino acid residues from the reference constituent polypeptide, at least 8, 10, 15, 20, 30 or 40 amino acids, and often has at least 50, 75, 100, 200, 300, 400 or more amino acids of a polypeptide of the invention, up to the full length polypeptide minus one amino acid. The appropriate length and amino acid sequence of a functional fragment of a constituent polypeptide of the invention can be determined by those skilled in the art, depending on the intended use of the functional fragment. For example, a functional fragment of anastellin (SEQ ID NO: 1) is intended to refer to a portion of anastellin that still retains some or all of the fibronectin or fibrinogen polymerizing activity of the reference polypeptide. Therefore, a functional fragment of anastellin, antithrombin, endostatin, or anginex can contain at least one or more binding sites necessary for acting in concert with fibronectin or vitronectin to effect angiogenesis inhibition.

Alternatively, a functional fragment of anastellin, antithrombin, endostatin, or anginex can contain that part of the amino acid sequence of the reference polypeptide required for inhibition of angiogenesis, tumor growth or metastasis. Similarly, a functional fragment of fibronectin, vitronectin, or fibrinogen can contain at least one or more binding sites necessary for aggregation by a polymerizing agent.

Minor modifications in the primary amino acid sequence of anastellin, antithrombin, endostatin, anginex, fibronectin, superfibronectin, and vitronectin can result in polypeptides that retain substantially equivalent function. These modifications can be deliberate, as through site-directed mutagenesis, or can be accidental such as through spontaneous mutation. For example, it is understood that only a portion or fragment of anastellin, antithrombin, endostatin, or anginex can form an angiogenesis-inhibiting compound with fibronectin, vitronectin, or fibrinogen. Conversely, only a portion or fragment of fibronectin, vitronectin, or fibrinogen can be incubated with anastellin antithrombin, endostatin, or anginex, respectively, to produce an angiogenesis-inhibiting compound. Similarly, a portion or fragment of anastellin, antithrombin, endostatin, or anginex that retains functional activity with regard to inhibition of angiogenesis is also encompassed by an angiogenesis inhibitor useful in the compositions and methods of the invention. It is understood that the various constituent polypeptides and compositions can be attached to a polypeptide of the invention, for example, other polypeptides, carbohydrates, lipids, chemical moieties or polymerizing agents.

The constituent polypeptides of the compositions and methods of the invention, or any fibronectin, vitronectin, or fibrinogen polymerizing agent that retains at least one of the functional activities described herein, can be isolated or synthesized using methods well-known in the art. Such methods include recombinant DNA methods and chemical synthesis. Anastellin, antithrombin, endostatin, anginex, fibronectin, fibronectin fragments, vitronectin, vitronectin fragments, fibrinogen, fibrinogen fragments or any other constituent polypeptide of the invention can be isolated from animal tissue or plasma or produced and isolated from cell culture as well as from genetically altered animals, such as transgenic animals. Methods that can be used in synthesizing fibronectin or fibronectin fragments or modifications useful for generating superfibronectin are well-known in the art, and include those described in Morla et al., supra (1994).

The constituent polypeptides of the invention and fragments thereof can be purified by a variety of methods well-known in the art, including recombinant expression systems described herein, precipitation, gel filtration, ion-exchange, reverse-phase and affinity chromatography, and the like. Other well-known methods are described in Deutscher et al., Methods in Enzymology Vol. 182, “Guide to Protein Purification” (Academic Press 1990), which is incorporated herein by reference. Alternatively, the constituent polypeptides of the invention can be obtained using well-known recombinant methods as described, for example, in Sambrook et al., Molecular Cloning: A Laboratory Manual (2d ed., Cold Spring Harbor Laboratory Press, New York 1989) and Ausubel et al., Current Protocols in Molecular Biology (John Wiley & Sons, New York 2000).

The methods and conditions for biochemical purification of a constituent polypeptide of the invention or fragment thereof can be chosen by those skilled in the art, and purification monitored, for example, by gel electrophoresis, an immunological assay, a binding assay, or a functional assay. For example, anastellin, antithrombin, endostatin, anginex, fibronectin, vitronectin, and fibrinogen as well as fragments of these polypeptides can be synthesized or obtained from plasma, cultured cells or any tissue source by methods well-known in the art for protein isolation and purification. Constituent polypeptides of the invention and fragments thereof obtained from cultured cells can be natural or recombinant polypeptides. Furthermore, anastellin, endostatin, antithrombin III, fibronectin, superfibronectin, vitronectin and fibrinogen are commercially available from a variety of sources including, for example, Sigma Aldrich, St. Louis, Mo.; Calbiochem, La Jolla, Calif.; and Chemicon, Temecula, Calif.

Methods for chemical and proteolytic cleavage and for purification of the resultant protein fragments are well-known in the art (see, for example, Deutscher, supra (1990)), which is incorporated herein by reference). For example, a chemical such as cyanogen bromide or a protease such as trypsin, chymotrypsin, V8 protease, endoproteinase Lys-C, endoproteinase Arg-C or endoproteinase Asp-N can be used to produce fragments of the constituent polypeptides of the invention.

Methods for quantitative analysis of samples containing constituent polypeptides of compositions of the invention to determine the amount of a constituent polypeptide or composition of the invention are well-known in the art and include absorption measurements in the ultraviolet and in the visibility range by direct or colorimetric protein determination. These methods are useful, for example, to determine the amount of polymeric fibronectin or fibrinogen formed upon incubation of fibronectin or fibrinogen, respectively, with a polymerizing agent such as anastellin antithrombin, endostatin, or anginex, include optical density measurement (Pasqualini et al., supra (1996); Yi and Ruoslahti, supra (2001)) or dynamic light scattering. An appropriate method for protein quantification can be selected based on a variety of factors well-known in the art, including protein purity and amount of sample.

Thus, the invention provides substantially pure compositions of anastellin, antithrombin, endostatin, anginex, as well as substantially pure compositions of their complexes with fibronectin, vitronectin, or fibrinogen. The invention also provides methods of inhibiting angiogenesis, metastasis and tumor growth by administering angiogenesis inhibitors along with RGD-containing plasma adhesion proteins in a pharmaceutically acceptable carrier. Angiogenesis inhibitors useful in this invention include, but are not limited to, anastellin, antithrombin, endostatin, anginex, or other compositions described herein.

Embodiments of the invention further provide a method of inhibiting angiogenesis by administering angiogenesis inhibitors along with an RGD-containing plasma adhesion protein in an amount effective to inhibit angiogenesis, where the amount of angiogenesis inhibitor is 0.05 mg or greater, as well as a method of inhibiting tumor growth by administering angiogenesis inhibitors in an amount effective to inhibit angiogenesis, where the amount is 0.05 mg or greater. The invention also provides a method of inhibiting tumor growth by administering angiogenesis inhibitors in an amount effective to inhibit metastasis, where the amount is 0.05 mg or greater. Angiogenesis inhibitors useful in this invention include, but are not limited to anastellin, antithrombin, endostatin, anginex, or other compositions described herein.

As used herein, the term “effective amount” when used in reference to methods for inhibiting angiogenesis, is intended to mean any reduction in the growth of blood vessels or in the neo-vascularization or re-vascularization of a tissue when compared to treatment with an inactive control compound or absence of treatment. Furthermore, as used herein, the term “effective amount” in reference to methods for inhibiting tumor growth is intended to mean the amount of a composition or polypeptide of the invention that can reduce the number, size or proliferation of neoplastic cells when compared to treatment with an inactive control compound or absence of treatment. Similarly, when used in reference to methods for inhibiting metastasis, the term “effective amount” is intended to mean any reduction in the movement of tumor cells from a primary site by any route, any decrease in the number of circulating tumor cells, any increase in the removal of tumor cells from the circulation, or any reduction in the occurrence of neoplastic growth at secondary sites when compared to treatment with an inactive control compound or absence of treatment.

The actual amount considered to be an effective amount for a particular application can depend, for example, on such factors as the affinity, avidity, stability, bioavailability, or selectivity of the molecule, as well as the moiety attached to the molecule, the pharmaceutical carrier, and the route of administration. Effective amounts can be determined or extrapolated using methods known to those skilled in the art. Such methods include, for example, in vitro assays with cultured cells or tissue biopsies and animal models known to those skilled in the art. For example, an appropriate amount and formulation for inhibiting tumor growth, metastasis or angiogenesis in humans can be extrapolated based on testing the efficacy of the compound in an animal model. By testing a spectrum of different dosage amounts, an optimum dosage can be determined and extrapolated for administration to a human subject.

The growth of solid tumors and the metastatic process is dependent on tumor angiogenesis. In humans, a tumor which is not able to stimulate its own vascularization can for years be restricted in growth to a microscopic region and limited to a million or less cells in size. Stimulation of blood vessel growth is a prerequisite of the conversion of a tumor to an angiogenic phenotype and involves a change in the local balance of blood vessel growth inhibitors and growth stimulators. In addition to allowing a tumor to increase in size, vascularization provides a means for tumor cell metastasis. The methods of the invention are useful in treating the types of cancer that exhibit angiogenesis, solid tumor growth and metastasis. Tumor types that are susceptible to treatment with the methods provided by the invention include, for example, epithelial cancers such as breast cancer, melanomas, sarcomas (Example I), lymphomas, and leukemias.

The compound and methods of this invention are also useful in non-malignant diseases associated with abnormal angiogenesis. Such diseases include rheumatoid arthritis and other inflammatory conditions, macular degeneration of the eye, and atherosclerosis.

As shown in the Examples that follow, anastellin, antithrombin, and endostatin can inhibit angiogenesis. It is likely that the low number of blood vessels is an impediment to tumor growth, given that vascularization is a prerequisite for tumor growth as described in Hanahan and Folkman, Cell 86:353-364 (1996). A related decrease in metastasis is likely. These anti-tumor effects have been shown for anastellin and its combination with fibronectin (superfibronectin) (Pasqualini et al., supra (1996); Yi and Ruoslahti, supra (2001)).

Inhibition of angiogenesis is shown in Examples I-V, namely inhibition of angiogenesis by anastellin and fibronectin, described in Examples II and III, antithrombin and vitronectin, described in Example IV, and endostatin and fibronectin, described in Example V. Examples III-V demonstrate a dependency on the RGD-containing plasma adhesion proteins for anti-angiogenic activity.

The compositions of the invention can be formulated and administered by those skilled in the art in a manner and in an amount appropriate for the nature of the pathology to be treated; the weight, gender, age and health of the subject; the biochemical nature, bioactivity, bioavailability and side effects of the particular composition; and in a manner compatible with concurrent treatment regimens. For example, an appropriate amount and formulation for inhibiting tumor growth or angiogenesis in humans can be extrapolated from animal models known to those skilled in the art based on the particular disorder. It is understood, that the dosage of a composition administered to a subject should be adjusted based on the bioactivity of the composition as well as on the metabolic characteristics of the subject. Therefore, once an optimum dosage has been determined based on testing a spectrum of different dosage amounts in an animal model, the optimum dosage amount can be extrapolated for administration to a human subject.

The compositions of the invention can be administered at various times based on the targeted results. It is understood that the timing for initiation of treatment can be determinative of the therapeutic results. In this regard, it is preferable to administer the compositions of the invention at an early stage of tumor growth so as to maximize the anti-angiogenic effects before large amounts of antagonistic angiogenic compounds are present. In addition, in order to prevent metastasis, sustained administration of the invention compositions can take place over a prolonged time.

The total amount of a composition of the invention can be administered as a single dose or by infusion over a relatively short period of time, or can be administered in multiple doses administered over a more prolonged period of time. Such considerations will depend on a variety of factors such as, for example, the state of the disease and context of the treatment regimen. For example, if the goal is to inhibit metastasis or tumor growth, the composition can be administered in a slow-release matrix, which can be implanted for systemic delivery or at the site of a desired target tissue. Contemplated matrices useful for controlled release of therapeutic compounds are well known in the art, and include materials such as DepoFoam™, biopolymers, micropumps, and the like. On the other hand, anastellin most effectively inhibits angiogenesis and tumor growth when administered in a single high dosage of 0.5 mg or greater. Based factors including, for example, tumor size and number of metastatic foci, several doses of 0.5 mg can be administered at predetermined time intervals.

The compositions can be administered to the subject by any number of routes known in the art including, for example, systemically, such as intravenously, intra-arterially, or intraperitoneally. A composition of the invention can be provided in the form of isolated and substantially purified polypeptides and polypeptide fragments in pharmaceutically acceptable formulations using formulation methods known to those of ordinary skill in the art. These formulations can be administered by standard routes, including for example, topical, transdermal, intraperitoneal, intracranial, intracerebroventricular, intracerebral, intravaginal, intrauterine, oral, rectal, or parenteral (e.g., intravenous, intraspinal, subcutaneous or intramuscular) routes. Preferred routes of administration that are particularly useful for administering the compositions of the invention include intraperitoneal and intravenous administration.

A composition can be administered as a solution or suspension together with a pharmaceutically acceptable carrier. Such a pharmaceutically acceptable carrier can be, for example, sterile aqueous solvents such as sodium phosphate buffer, phosphate buffered saline, normal saline or Ringer's solution or other physiologically buffered saline, or other solvent or vehicle such as a glycol, glycerol, an oil such as olive oil or an injectable organic ester. Superfibronectin can be prepared by mixing anastellin, endostatin or anginex with fibronectin, or antithrombin with vitronectin, in a buffer that is appropriate for subsequent administration in vivo. A pharmaceutically acceptable carrier can additionally contain physiologically acceptable compounds that act to, for example, stabilize the composition or increase its absorption. Such physiologically acceptable compounds include, for example, carbohydrates such as glucose, sucrose or dextrans; antioxidants such as ascorbic acid or glutathione; receptor mediated permeabilizers, which can be used to increase permeability of the blood-brain barrier; chelating agents such as EDTA, which disrupts microbial membranes; divalent metal ions such as calcium or magnesium; low molecular weight proteins; lipids or liposomes; or other stabilizers or excipients. Those skilled in the art understand that the choice of a pharmaceutically acceptable carrier depends on the route of administration of the compound containing the neutralizing agent and on its particular physical and chemical characteristics.

Formulations suitable for parenteral administration include aqueous and non-aqueous sterile injection solutions such as the pharmaceutically acceptable carriers described above. The solutions can additionally contain, for example, buffers, bacteriostats and solutes that render the formulation isotonic with the blood of the intended recipient. Other formulations include, for example, aqueous and non-aqueous sterile suspensions that can include suspending agents and thickening agents. The formulations can be presented in unit-dose or multi-dose containers, for example, sealed ampoules and vials, and can be stored in a lyophilized condition requiring, for example, the addition of the sterile liquid carrier, immediately prior to use. Extemporaneous injection solutions and suspensions can be prepared from sterile powders, granules and tablets of the kind previously described.

A constituent polypeptide or composition of the invention can be incorporated into a material that allows for sustained release of the composition useful for inhibiting tumor growth, angiogenesis or metastasis. The sustained release form has the advantage of inhibiting growth, metastases, endothelial growth or the like over an extended period of time without the need for repeated administrations. Sustained release can be achieved, for example, with a sustained release material such as a wafer, an immunobead, a micropump or other material that provides for controlled slow release. Such controlled release materials are well-known in the art and available from commercial sources (Alza Corp., Palo Alto Calif.; Depotech, La Jolla Calif.; see also Pardoll, Ann. Rev. Immunol. 13:399-415 (1995), which is incorporated herein by reference). In addition, biodegradable polymers and their use are described, for example, in Brem et al., J. Neurosurg. 74:441-446 (1991), which is incorporated herein by reference. In addition, a bioerodible or biodegradable material that can be formulated with anastellin or any of the compositions of the invention, such as polylactic acid, polygalactic acid, regenerated collagen, multilamellar liposomes or other conventional depot formulations, can be implanted to slowly release anastellin or a particular composition of the invention. The use of infusion pumps, matrix entrapment systems, and transdermal delivery devices also are contemplated in the present invention.

The compositions also can be advantageously enclosed in micelles or liposomes. Liposome encapsulation technology is well known. Liposomes, which consist of phospholipids or other lipids, are nontoxic, physiologically acceptable and metabolizable carriers that are relatively simple to make and administer, and can be targeted to a specific tissue, such as neural tissue, through the use of receptors, ligands or antibodies capable of binding the targeted tissue. The technology and preparation of such formulations is well known in the art, see, for example, Radin, et al., Meth. Enzymol. 98:613-618 (1983); Gregoriadis, Liposome Technology Vols. I to III, (2d ed., CRC Press, Boca Raton Fla. 1993) and Nabel et al., Proc. Natl. Acad. Sci. USA 90:11307-11311 (1993), which are incorporated herein by reference. It is understood that liposomes are desirable for applications that require an increase in the lipophilicity of the compound such as those applications that involve crossing of the blood-brain barrier.

Embodiments of the invention also include methods in which anastellin, antithrombin, endostatin, anginex, or their complexes with fibronectin or vitronectin are generated in vivo. These methods include implanting into the subject a cell genetically modified to express and secrete anastellin, antithrombin, endostatin, anginex, or any of the constituent polypeptides in vivo. The invention methods also encompass gene therapy involving inserting into the subject genes that are capable of expressing anastellin, antithrombin, endostatin, anginex, or any of the constituent polypeptides in vivo. For a subject suffering from a long-term risk of metastasis or tumor recurrence, such methods have the advantage of obviating or reducing the need for repeated administration.

For ex vivo gene transfer, using methods well-known in the art, a cell can be transiently or stably transfected with an expression vector containing the desired nucleic acid sequences, for example as described in Chang, Somatic Gene Therapy (CRC Press, Boca Raton 1995), which is incorporated herein by reference. The transfected cell is then implanted into the subject. Methods of transfecting cells ex vivo are well known in the art, see, for example, Kriegler, Gene Transfer and Expression: A Laboratory Manual (W.H. Freeman & Co., New York 1990), incorporated herein by reference. For the transfection of a cell that continues to divide such as a fibroblast, muscle cell, glial cell or neuronal precursor cell, retroviral or adenoviral vectors can be used. For the transfection of a nucleic acid into a postmitotic cell such as a neuron, for example, a replication defective herpes simplex virus type 1 or Sindbis virus vector can be used, and such methods are well-known in the art, as in During et al., Soc. Neurosci. Abstr. 17:140 (1991); Sable et al., Soc. Neurosci. Abstr. 17:570 (1991); Dubensky et al., J. Virology 70:508-519 (1996), each of which is hereby incorporated by reference.

For in vivo gene therapy, using methods well-known in the art, the desired cell or tissue can be transiently or stably transfected with an expression vector containing the desired nucleic acid sequence(s) to effect expression of anastellin, antithrombin, endostatin, anginex, or any of the constituent polypeptides of the invention in vivo, for example, as described in Acsadi et al., New Biol. 3:71-81 (1991); Chang, supra (1995); Chen et al., Proc. Natl. Acad. Sci. USA 91:3054-3057 (1994); Culver et al., Science 256:1550-1552 (1992); Furth et al., Molec. Biotech. 4:121-127 (1995); all of which are hereby incorporated by reference.

In current cancer treatment regimes, more than one compound is often administered to an individual for management of the same or different aspects of the disease. Thus, for use in inhibiting angiogenesis, tumor growth or metastasis, a composition of the invention can advantageously be formulated with a second compound such as a antineoplastic agent such as, for example, tamoxifen, doxorubicin or cyclophosphamide, as well as with compounds administered to reduce side-effects of antineoplastic agents. Contemplated methods of inhibiting tumor growth, metastasis and angiogenesis include administering a compound of the invention alone, in combination with, or in sequence with, such other compounds. Alternatively, combination therapies can consist of fusion proteins, where a constituent polypeptide of a composition of the invention is linked to a heterologous protein, such as a therapeutic protein or targeting protein. Heterologous proteins useful for practicing this embodiment of the invention include, for example, RGD peptides. The compositions of the invention can be administered as part of a treatment regimen that includes, for example, radiation, chemotherapy, antibody therapy or any combination of these and other therapies.

It is understood that modifications which do not substantially affect the activity of the various embodiments of this invention are also included within the definition of the invention provided herein. Accordingly, the following examples are intended to illustrate but not limit the present invention.

### EXAMPLE I

### Anastellin Alone or Combined with Fibronectin Ex Vivo Inhibits Tumor Angiogenesis

Anastellin and its complexes with fibronectin (superfibronectin) inhibit tumor growth upon systemic administration to mice bearing various types of tumors (Yi and Ruoslahti, supra 2001). This example describes inhibition of tumor angiogenesis by anastellin and superfibronectin.

Anastellin and III₁₁-C, a control fibronectin fragment from type III repeat 11, were prepared as recombinant his-tagged proteins in bacteria and purified as described in Morla et al., supra (1994), and Pasqualini et al., supra (1996). Human plasma fibronectin was obtained from Chemicon (Temecula, Calif.) and human fibrinogen was obtained from Sigma (St. Louis, Mo.). Fibronectin was converted to superfibronectin by mixing 100 μg fibronectin in 100 μl PBS with 300 μg anastellin in 100 μl PBS as described in Pasqualini et al., supra (1996). Protein solutions were sterilized by filtering through 0.2 μm membrane prior to polymerization.

The MDA-MB-435 breast cancer human tumor cell lines were cultured and harvested and used to establish human xenograft tumors in nude mice as described in Pasqualini et al., supra (1996) and Arap et al., Science 279:377-380 (1998), which are incorporated herein by reference. Briefly, the cells were allowed to grow in the continuous culture for no more than three consecutive passages before being used in the experiments. Actively growing cells were detached from culture plates with PBS/2.5 mM EDTA or Trypsin-EDTA (0.25% trypsin, 1 mM Na-EDTA; Gibco BRL, Rockville, Md.). The detached cells were resuspended in DMEM, counted and examined for viability by trypan blue exclusion. Subsequently, the cells were injected into mice as described below. A portion of the cells used in the injections was seeded back into a culture plate to determine plating efficiency. The viability was higher than 99% and the plating efficiency greater than 95%.

The tumor cells were injected into two-month old immunodeficient Balb/c/nu/nu, female mice (Harlan Sprague-Dawley, San Diego, Calif.). Briefly, to obtain subcutaneous tumors, 10⁶ tumor cells suspended in 200 μl of DMEM were injected into the right posterior flank of the mice, which were randomized and divided into experimental groups of 5-6 mice per group. At 3 weeks after tumor cell implantation nearly all of the mice had developed palpable tumors. The mice were treated with intraperitoneal injections of either anastellin or superfibronectin in PBS, or with PBS alone. The treatments were administered twice a week via intraperitoneal injections for the duration of the study. The injections were given in 200 μl of PBS. Subcutaneous tumors were grown in nude mice from MDA-MB-435 breast cancer cells cultured and harvested as described above. Treatments with biweekly intraperitoneal injections of 6 mice per treatment group of either anastellin or superfibronectin were started three weeks after tumor implantation and continued for 5 weeks. Each injection consisted of 600 μg of anastellin or 100 μg of fibronectin mixed with 300 μg of anastellin. In some experiments unpolymerized fibronectin (100 μg per injection) or the III₁₁-C fragment of fibronectin (600 μg per injection) were used as additional controls. At about 8 weeks after tumor cell implantation, which corresponds to about 5 weeks after the start of the treatments, the mice were anesthetized and perfused through the heart with PBS.

To determine whether the inhibition of tumor growth (Yi and Ruoslahti, supra (2001)) is due to inhibition of tumor angiogenesis, blood vessel density was determined using sections of tumors collected at the termination of the tumor growth inhibition studies described above. Paraffin embedding, sectioning and immunostaining for blood vessels with anti-CD31 (rat anti-mouse, Pharmingen, San Diego, Calif.) were carried out in The Burnham Institute Histology Facility.

Reduced Tumor Angiogenesis in MDA-MB-435 Breast Cancer Tumors

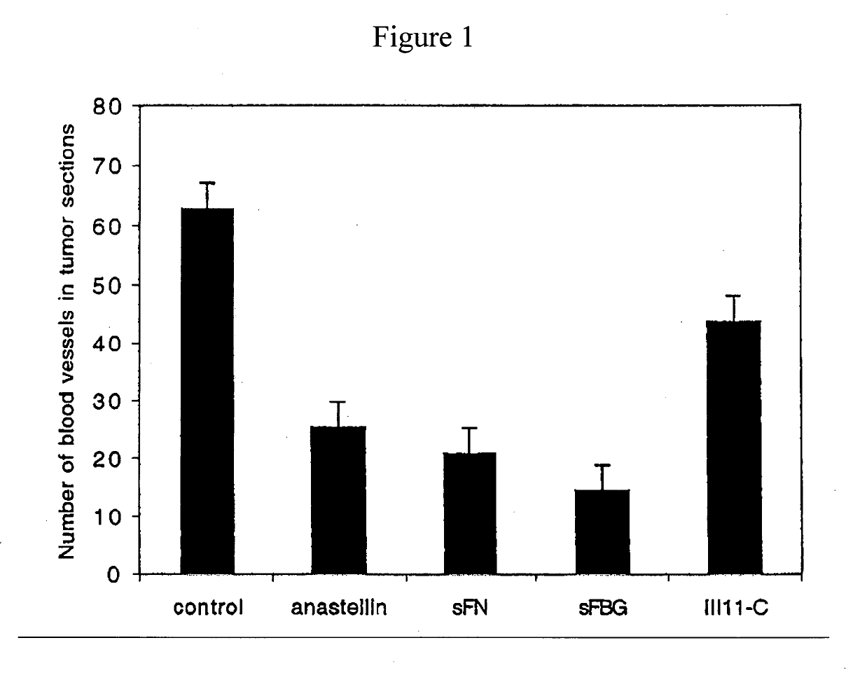

MDA-MB-435 breast cancer tumors from a tumor growth inhibition study similar to the one described above were removed at the termination of the study, sectioned, and the sections were stained with anti-CD31 antibodies to visualize tumor blood vessels. Representative microscopic fields from the tumors showed higher density of blood vessels in the vehicle alone group than in the anastellin, anastellin plus fibronectin (superfibronectin), and anastellin groups (see FIG. 1). Similar results were obtained with KRIB human osteosarcoma and C8161 human melanoma xenograft tumors. Tumor growth and metastasis were inhibited in mice treated with anastellin, and by anastellin plus fibronectin (Yi and Ruoslahti, supra (2001)).

### EXAMPLE II

Anastellin in Conjunction with Fibronectin Inhibits Angiogenesis

This example describes the effects of systemically administered anastellin on angiogenesis.

Matrigel Angiogenesis Assay

To study anastellin as an angiogenesis inhibitor, a non-tumor angiogenesis model was used. Basement membrane material (matrigel) was impregnated with angiogenic factors and implanted into mice to induce angiogenesis that rapidly supplies the plug with vasculature (Fulgham et al., Endothelium 6(3):185-195 (1999); Ngo et al., Cell Growth Differ 11(4):201-210 (2000)). Matrigel was from Becton Dickinson, (Bedford, Mass.). Recombinant human bFGF and recombinant mouse VEGF were from R&D Systems, (Minneapolis, Minn.). The rat anti-mouse CD31 antibody was from Pharmingen, (San Diego, Calif.). Liquid matrigel containing 100 ng of bFGF or 50 ng of VEGF per ml was injected subcutaneously in the abdominal region of the mouse. Each mouse received one or two 0.5 ml matrigel plugs. The mice were treated with daily intraperitoneal injections of one of the angiogenesis inhibitors or PBS as a control. In some experiments, a fragment corresponding to the homologous residues from the 11^{th} type III domain of human fibronectin was used at the same dose as anastellin to provide an additional treatment control for anastellin (Pasqualini et al., supra (1996)). After one week, the mice were sacrificed and the matrigel plugs removed. Half of the matrigel plugs were homogenized and their hemoglobin content was determined using the Drabkin reagent kit (Sigma). The remaining plugs were fixed in 4% paraformaldehyde and stored in 70% ethanol. Paraffin embedding, sectioning and immunostaining of the plugs for CD31 and other blood vessel markers were carried out in The Burnham Institute Histology Facility or at Pharmingen (La Jolla, Calif.). An average of three sections were examined from each matrigel plug. Student's T-test was used in statistical analysis of the results. The hemoglobin assay tended to have less experimental variation than the blood vessel counts, presumably because the hemoglobin content of an entire matrigel plug was studied, whereas blood vessels were counted from a limited number of histological sections.

Anastellin Inhibits Angiogenesis Stimulated by bFGF and VEGF

Both bFGF and VEGF stimulated matrigel angiogenesis, and the number of blood vessels in the plugs correlated with the amount of the angiogenic factor added to the gel. Based on these experiments, 100 ng of bFGF or 50 ng were used as the angiogenic stimulus for the testing of angiogenesis inhibitors.

Mice bearing matrigel plugs impregnated with bFGF were treated with daily intraperitoneal injections of 1 mg of anastellin in 0.3 ml of PBS, a control fragment homologous to anastellin from the 11^{th} type III domain of fibronectin (1 mg in PBS), or PBS. The treatment was continued for 10 days. Angiogenesis was evaluated by measuring the hemoglobin content of the plugs, and by counting the number of blood vessels in tissue sections stained for blood vessel marker CD31 in duplicate plugs. Anastellin almost completely inhibited matrigel plug angiogenesis induced by bFGF or VEGF, but did not significantly affect the low level of vascularization in plugs that received no growth factor. The control fragment homologous to anastellin but derived from another (11^{th}) fibronectin type III domain was inactive.

### EXAMPLE III

Plasma Fibronectin is Necessary for the Anti-Angiogenic Effects of Anastellin

To test the hypothesis that the interaction of anastellin with fibronectin would be critical to angiogenic activity of anastellin, mutant mice that conditionally lack plasma fibronectin (Sakai et al., Nature Med. 7:324-330 (2001 0) were used.

Two-month old immunodeficient Balb/c/nu/nu, female mice (Harlan Sprague-Dawley, San Diego, Calif.), wild type C57BL/6J mice, and transgenic mice were used for the experiments.

Two plasma fibronectin Cre/loxP conditional knockout mouse lines have been described (Sakai et al., supra (2001)). In one of these lines, Cre expression is under the control of the albumin promoter and causes postnatal elimination of the fibronectin gene in the liver, which is the source of essentially all of plasma fibronectin. The other line expresses Cre in an interferon-inducible manner. These mice have been shown to express less than 0.04% of the normal plasma fibronectin level (Sakai et al., supra (2001)). The mice were genotyped, and their plasma fibronectin level was examined by immunoblotting.

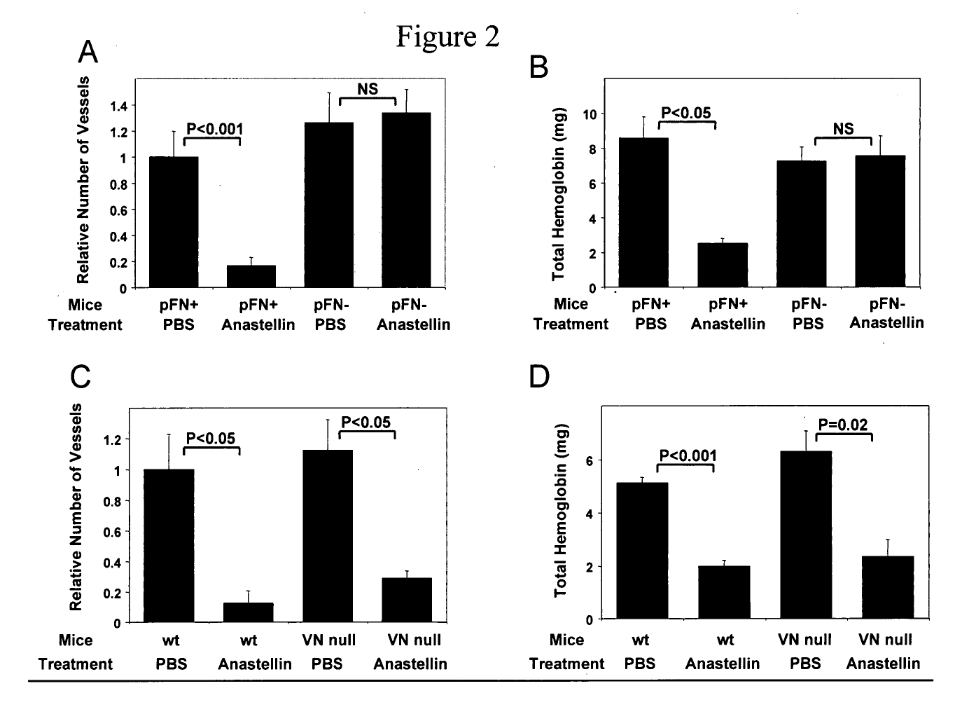

Fibronectin-deficient mice (pFN−) and their normal littermates (pFN+) with matrigel plugs were treated with seven daily injections of 1 mg of anastellin as described above. Angiogenesis was evaluated by counting the number of blood vessels in tissue sections from the plugs stained for the blood vessel marker CD31 (FIGS. 2A and C), and by measuring the hemoglobin content of duplicate plugs (FIGS. 2B and D). The two fibronectin-deficient lines gave similar results; these results were combined in panels A and B (56 mice were used in 5 independent experiments). Anastellin had no anti-angiogenic activity in the fibronectin-deficient mice, but was fully active in the normal littermates of these mice (FIGS. 2A and B). Anastellin was fully active in vitronectin null (VN null) mice and wild type control (wt) mice of the same strain as the null mice (FIGS. 2C and D). The vitronectin null mice (Zheng et al., Proc. Natl. Acad. Sci. USA 92:12426-12430 (1995)) were obtained from the Scripps Research Institute (San Diego, Calif.) and bred and maintained in the Burnham Institute animal facility. The mice were genotyped, and their vitronectin levels examined by immunoblotting. These results show that plasma fibronectin, but not vitronectin, is required for anastellin to be anti-angiogenic.

### EXAMPLE IV

Vitronectin is Necessary for Anti-Angiogenic Activity of Antithrombin

This example describes the anti-angiogenesis effects of systemically administered antithrombin in the plasma fibronectin-deficient (pFN−) and vitronectin null (VN null) mice. Antithrombin modified by denaturation or proteolysis is an angiogenesis inhibitor (O'Reilly et al., supra (1999)), and antithrombin modified in this manner also binds to vitronectin (Ill and Ruoslahti, supra (1985); deBoer et al. supra (1992)).

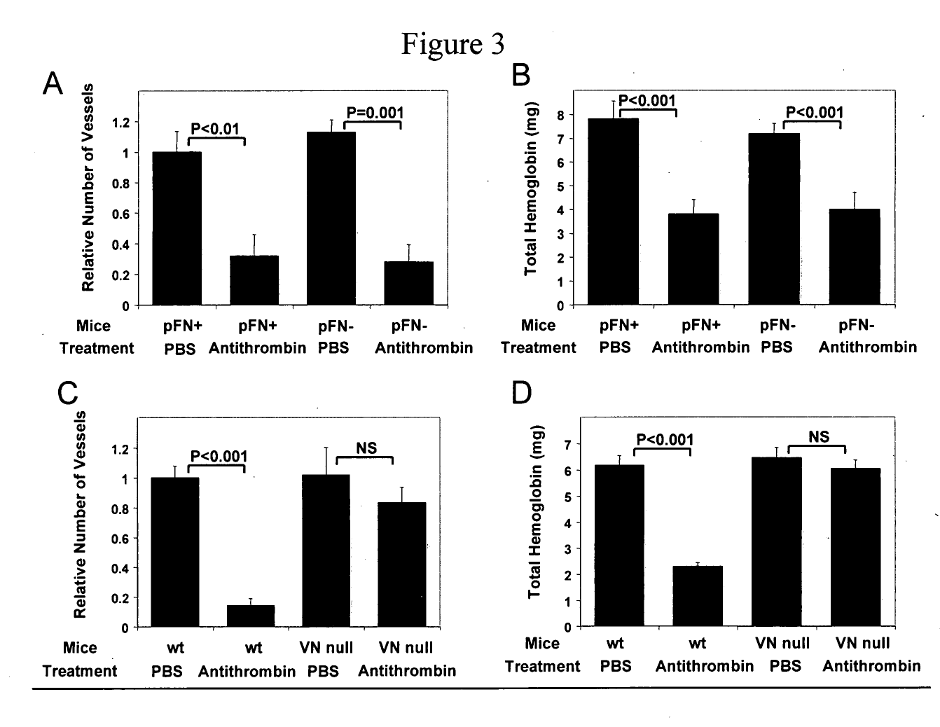

To test the dependence of the antithrombin anti-angiogenic activity on plasma fibronectin and vitronectin, fibronectin-deficient mice (pFN−), as described in Example III, their wild type littermates (pFN+), and vitronectin null (null) mice and their wild type controls (wt) were implanted with matrigel plugs and systemically treated with 7 daily intraperitoneal injections of 180 or 270 micrograms of antithrombin in 0.3 ml of PBS, or with PBS as described for anastellin in Example II. Angiogenesis was evaluated by counting the number of blood vessels in tissue sections from the plugs stained for the blood vessel marker CD31 (FIGS. 3A and C), or by measuring the hemoglobin content of duplicate plugs (FIGS. 3B and D). Denatured antithrombin inhibited angiogenesis in fibronectin-deficient (pFN−) mice, their wild type littermates (pFN+) and in the wild type controls (wt) for the vitronectin null mice, but was inactive in the vitronectin null (VN null) mice (FIG. 3). These results show that vitronectin is required for the anti-angiogenic activity of antithrombin, but fibronectin is not.

### EXAMPLE V

Fibronectin is Necessary for the Anti-Angiogenic Activity of Endostatin

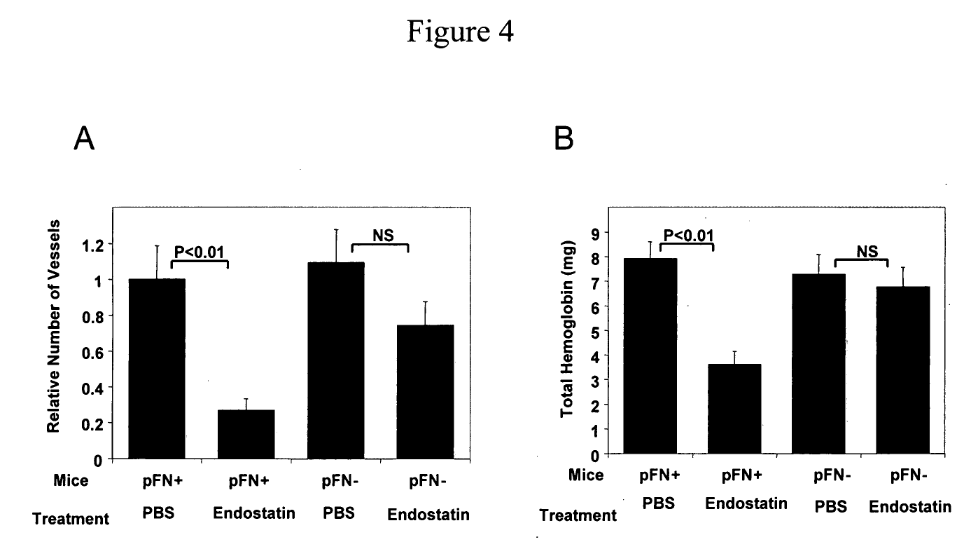

Like anastellin, endostatin is an angiogenesis inhibitor that is derived from an extracellular matrix protein. To test the dependence of the endostatin anti-angiogenic activity on plasma fibronectin, fibronectin-deficient mice (pFN−), as described in Example III, and their wild type littermates (pFN+) were implanted with matrigel plugs and treated with seven (7) daily injections of 120 micrograms of endostatin as described for anastellin in Example III. Angiogenesis was evaluated by counting the number of blood vessels in tissue sections from the plugs stained for the blood vessel marker CD31 (FIG. 4A), or by measuring the hemoglobin content of duplicate plugs (FIG. 4B). Endostatin was inactive in the fibronectin-deficient mice (FIG. 4) but active in their normal littermates.

### EXAMPLE VI

Anginex Polymerizes Fibronectin

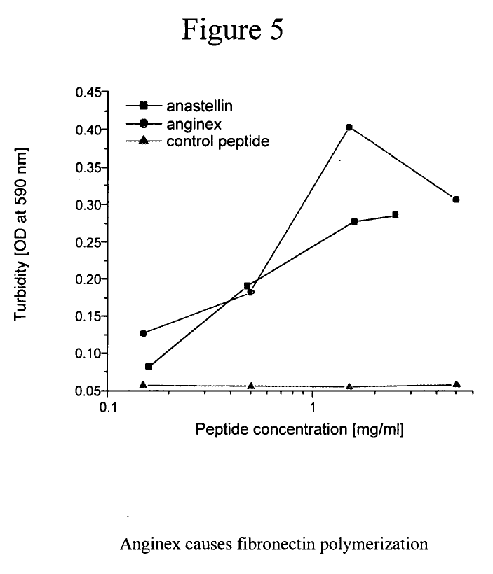

Increasing concentrations of anginex, anastellin (positive control), or an unrelated peptide (negative control) were mixed with constant amount of a fibronectin solution in phosphate-buffered saline to give a final concentration of 0.5 mg/ml. The samples were incubated at room temperature and the optical density at 590 nm. FIG. 5 shows turbidity resulting from polymer formation at the 3-hour time point after the proteins were mixed. These results show that anginex is similar to anastellin in being able to polymerize fibronectin.

Throughout this application various publications have been referenced within parentheses. The disclosures of these publications in their entireties are hereby incorporated by reference in this application to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A substantially pure composition comprising an angiogenesis inhibitor and vitronectin in a pharmaceutically acceptable carrier.

2. The composition of claim 1, wherein the angiogenesis inhibitor comprises a polypeptide having at least 90% sequence identity to antithrombin (SEQ ID NO:2).

3. The composition of claim 1, wherein the angiogenesis inhibitor comprises a polypeptide having at least 95% sequence identity to antithrombin (SEQ ID NO:2).

4. The composition of claim 1, wherein the angiogenesis inhibitor comprises antithrombin (SEQ ID NO: 2), or a functional fragment thereof.

5. The composition of claim 1, wherein the angiogenesis inhibitor consists essentially of antithrombin (SEQ ID NO: 2), or a functional fragment thereof.

6. The composition of claim 1, wherein the angiogenesis inhibitor comprises endostatin.

7. A method of inhibiting angiogenesis in a patient, comprising:
providing a patient in need of angiogenesis-inhibiting treatment; and
administering to said patient an angiogenesis inhibitor and vitronectin.

8. The method of claim 7, wherein said angiogenesis inhibitor has at least 90% sequence identity to antithrombin (SEQ ID NO: 2).

9. The method of claim 7, wherein said angiogenesis inhibitor has at least 95% sequence identity to antithrombin (SEQ ID NO: 2).

10. The method of claim 7, wherein said angiogenesis inhibitor comprises antithrombin (SEQ ID NO: 2), or a functional fragment thereof.

11. The method of claim 7, wherein said angiogenesis inhibitor consists essentially of antithrombin (SEQ ID NO: 2) or a functional fragment thereof.

12. The method of claim 7, wherein the angiogenesis inhibitor comprises endostatin.

13. The method of claim 7, wherein the angiogenesis inhibitor is provided in an amount greater than 0.05 mg.

14. The method of claim 7, wherein the angiogenesis inhibitor and vitronecitn are provided simultaneously.

15. The method of claim 7, wherein the angiogenesis inhibitor and vitronectin are provided sequentially, in either order.

16. A method of inhibiting angiogenesis in a patient; comprising:
providing a patient in need of angiogenesis-inhibiting treatment;
determining the level of vitronectin in said patient; and
administering to said patient an angiogenesis inhibitor that is activated by vitronectin.

17. The method of claim 16, wherein said angiogenesis inhibitor has at least 90% sequence identity to antithrombin (SEQ ID NO: 2).

18. The method of claim 16, wherein said angiogenesis inhibitor has at least 95% sequence identity to antithrombin (SEQ ID NO: 2).

19. The method of claim 16, wherein said angiogenesis inhibitor comprises antithrombin (SEQ ID NO: 2), or a functional fragment thereof.

20. The method of claim 16, wherein said angiogenesis inhibitor consists essentially of antithrombin (SEQ ID NO: 2) or a functional fragment thereof.

21. The method of claim 16, wherein the angiogenesis inhibitor comprises endostatin.

22. The method of claim 16, wherein the angiogenesis inhibitor is provided in an amount greater than 0.05 mg.

23. The method of claim 16, wherein the angiogenesis inhibitor and vitronecitn are provided simultaneously.

24. The method of claim 16, wherein the angiogenesis inhibitor and vitronectin are provided sequentially, in either order.

25. The method of claim 16, further comprising administering to said patient an effective amount of vitronectin.

26. A method of treating cancer in a patient, comprising:
providing a patient in need of treatment of a tumor; and
administering to said patient an effective amount of angiogenesis inhibitor and vitronectin.

27. The method of claim 26, wherein said angiogenesis inhibitor has at least 90% sequence identity to antithrombin (SEQ ID NO: 2).

28. The method of claim 26, wherein said angiogenesis inhibitor has at least 95% sequence identity to antithrombin (SEQ ID NO: 2).

29. The method of claim 26, wherein said angiogenesis inhibitor comprises antithrombin (SEQ ID NO: 2), or a functional fragment thereof.

30. The method of claim 26, wherein said angiogenesis inhibitor consists essentially of antithrombin (SEQ ID NO: 2) or a functional fragment thereof.

31. The method of claim 26, wherein the angiogenesis inhibitor comprises endostatin.

32. The method of claim 26, wherein the angiogenesis inhibitor is provided in an amount greater than 0.05 mg.

33. The method of claim 26, wherein the angiogenesis inhibitor and vitronecitn are provided simultaneously.
